(19) United States
(12) Patent Application Publication
(10) Pub. No.: US 2013/0251661 A1
(43) Pub. Date: Sep. 26, 2013

(54) SOFTENER FOR TEXTILES

(71) Applicants: Jürgen Falkowski, Monheim (DE); Ansgar Behler, Bottrop (DE); Monika Barbenheim, Bottrop (DE)

(72) Inventors: Jürgen Falkowski, Monheim (DE); Ansgar Behler, Bottrop (DE); Monika Barbenheim, Bottrop (DE)

(21) Appl. No.: 13/993,945

(22) PCT Filed: Oct. 14, 2011
(86) PCT No.: PCT/EP2011/005158

§ 371 Date: Jun. 13, 2013

(30) Foreign Application Priority Data
Dec. 16, 2010 (EP) 10195396.6

(51) Int. Cl. A61K 8/55; C11D 3/00; C11D 3/36; A61Q 5/12

(52) U.S. Cl. CPC A61K 8/553; A61Q 5/12; C11D 3/001; C11D 3/365
USPC 424/70.21; 510/515

(57) ABSTRACT

What are described are textile treatment compositions comprising hydroxylated lecithin, at least one dispersant, preferably an alkyl(oligo)glycoside, at least one wax and water, and optionally further assistants and additives.

The present application relates to textile treatment compositions that comprise hydroxylated lecithin and to their use for the provision of soft handle in textiles or keratinic fibers, in particular hair.

Detergents and cleaning agents are produced in large amounts. The main application is the cleaning of fabrics and hard surfaces in the household and commercial domain.

Both in the production and processing of fabrics, as well as during washing in the domestic domain, the textiles in this process lose their original, usually soft, pleasant handle. This lies, for example, in the fact that the original fat or wax layers that are found, for example, on natural fibers such as cotton or sheep's wool, must be detached in several washing stages before further industrial processing. Therefore so-called softeners are applied again to industrially produced textiles in the last processing stage. This can be carried out—as is known to the person skilled in the art—by mechanical application (Foulard process) or alternatively in the extraction process. In the domestic field, these textiles have to be washed frequently for the removal of soiling. In this connection, surfactant-containing detergents and cleaning agents, which are always employed both in solid, usually as a powder or granules, or in liquid form.

This has the result that the softener originally applied to the textile is removed and the handle impaired. Furthermore, the deposition of alkali metal- or alkaline earth metal-containing residues, for example from the builder system, occurs during the washing process on the fibers, which render the fabric hard and stiff. Therefore in the domestic domain a liquid softener dispersion is frequently additionally added in the last rinsing operation of the washing machines.

This softener dispersion is then deposited in finely disperse form on the damp fabrics in the machine and is retained on the textiles after drying and ironing. This then leads to a soft, pleasant handle on the dry textiles, which improves the wearer comfort of the textiles or alternatively the use, for example, of hand or bath towels. Often, various perfume oils are admixed to the softener, which then provide, for example, for a fresh odor of the washed textiles. Furthermore, it is possible to apply these softeners to cloths which then transfer the softener to the drying textiles in the dryer at elevated temperature. In the case of the softeners employed in the household domain, these are usually products that are produced on the basis of amine and fatty acid compounds.

For improvement of the substantivity, in particular on cotton, these products usually contain a cationic charge, such as, for example, a quaternary ammonium compound. This charge moreover also brings about, in particular with textiles of synthetic material, an improvement in the antistatic properties of the dry fabrics. The best known representatives of this product class are the “ester quats” which are produced, for example, in a two-stage process by means of the esterification of triethanolamine with long-chain fatty acids and subsequent quaternization with dimethyl sulfate. Furthermore—in particular in the textile factories—aminolysis or amidation products of mono- or polyhydric amino alcohols with triglycerides or fatty acids are employed. Also known is distearyldimethyl-ammonium chloride, which was formerly frequently employed in household softeners, but today has been replaced by ester quats in almost all countries on account of the better biodegradability. A summary of the various textile softeners is found, for example, in the article “Softeners in Textile Processing” of Falkowski/Wahle, published 2002 in the journal Rev. Prog. Color 32 (2002).

A good compilation of household-close properties of these ingredients is also found, for example, in the handbook Wasch- und Reinigungsmittel [Detergents and Cleaning Agents] of Klaus Henning, published 2006 in Verlag VCI.

As mentioned above, one or more process steps are necessary for the production of the softener compounds customary today. In this connection, chemicals are also employed whose use necessitate increased safety technology requirements and that cannot be produced from renewable raw materials, such as, for example, polyamines or quaternization chemicals such as dimethyl sulfate. Furthermore, these types of softeners lead to a strong hydrophobization of the surface, that is the absorbability of the fabrics is impaired, which is undesired, for example, in the case of hand or bath towels.

It was therefore an object of the present invention to develop a product which produce improved sensory properties on textiles and that can simultaneously be produced based on renewable raw materials.

Lecithin is a known natural product, which can be obtained from animal or vegetable sources. In general, lecithin contains a nonpolar and a polar unit in the molecule. Furthermore, the molecule contains charged moieties, which bring about an outwardly neutral overall charge in the neutral pH range; in the acidic pH range the molecule is weakly cationic. Lecithins can therefore also be designated as zwitterions or internal salts. On account of the of the two different polar groups, the molecules are also surface-active. The nonpolar unit here probably brings about a plasticizing effect on soft surfaces, such as, for example, textiles. The cationic charge in the acidic range can improve the substantivity on cotton surfaces generally negatively charged during washing. Therefore it was also not surprising that lecithins are described in the literature as softeners for fibers or alternatively hair.

For example, U.S. Pat. No. 4,808,320 (Fabric softening Compositions based on lecithin and methods for making and using same; Jacques et al.; 1989, Colgate-Palmolive Company) describes the direct application of various concentrated lecithin dispersions as softeners for domestic washing. U.S. Pat. No. 8,161,70 (Stable aqueous fabric softening compositions based on lecithin, saponin and sorbic acid and methods for making and using same; Jacques et al.; 1989, Colgate-Palmolive Company) describes combinations of lecithins with saponins as softeners for domestic washing. U.S. Pat. No. 4,642,919 (Textile treating compositions and methods, Yi-Chang Fu, 1987 The Proctor & Gamble Company) describes combinations of lecithins with cationic softeners.

The processes described have some disadvantages, which are also the reason why these products are so far not obtainable commercially. Pure lecithin, as a natural product, is a highly viscous, brownish liquid, which can be processed only with difficulty. The production of pure lecithin dispersions or admixtures of these dispersions therefore likewise leads to unstable products, in particular difficult to meter at higher concentrations. Moreover, products are also too expensive for detergent and cleaning market. The applications have therefore so far remained restricted to the nutrition/food supplement area.

It has now been found that the use of hydroxylated lecithin can solve the problems described above.

A first subject of the present invention therefore relates to textile treatment compositions, comprising hydroxylated lecithin, at least one dispersant, at least one wax and water.

The production of the hydroxylated lecithins can be carried out, for example, by the treatment of the lecithin with hydrogen peroxide. In this connection, the double bond of the unsaturated fatty acid in the nonpolar molecule is oxidized and subsequently ring-opened using water. A production process for such lecithin derivatives and hydroxylated lecithins within the meaning of the present teaching are described, for example, in U.S. Pat. No. 2,621,133 or U.S. Pat. No. 6,638,544. U.S. Pat. No. 2,621,133 discloses the use of hydroxylated lecithin with starch or starch derivatives as sizing agents, but does not mention the use as textile softeners.

These hydroxylated lecithins are significantly lighter and more stable to oxidation than the non-hydroxylated lecithins. In this connection, fundamentally all types of vegetable or animal lecithins can be used, but preferably those qualities obtained based on ovalbumin, rapeseed oil or soya oil. An example of hydroxylated lecithin to be mentioned would be only, for example, the commercially obtainable product of the company Solae Company, Solec® A.

It has been found that these hydroxylated lecithins a) in combination with dispersants b) at room temperature (=21° C.) can disperse solid waxes c) in water. These dispersions according to the invention are light-colored, stable and highly liquid, which is important in particular for the metering and dispersion in a customary household washing machine. The sensory properties of the textiles treated with these products are better than the fabrics treated with customary textile softeners, e.g. by quaternary ammonium compounds. The compositions are liquids that contain solids in dispersed form. A dispersion, as is known, is a heterogeneous mixture of at least two substances, which cannot or can barely dissolve in one another or chemically combine with one another. Generally, these are colloids. Here, a substance (dispersed phase, disperse phase, inner phase or secondary phase) is dispersed as finely as possible in another substance (dispersing agent, dispersion medium, dispersant, continuous phase, outer phase or main phase). The individual phases can be delineated clearly from one another here.

According to the invention, the textile treatment agents have a kinematic viscosity (measured at 20° C. using a Brookfield viscometer, spindle 3, 20 rpm) of preferably less than/equal to 1000 mPa s. Particularly preferred are agents whose kinematic viscosity (measured at 20° C. using a Brookfield viscometer, spindle 3, 20 rpm) is between 15 and 250 mPa s, preferably between 25 and 150 mPa s, and in particular between 45 and 110 mPa s.

The dispersants b) employed can be non-ionic, anionic or cationic products.

Non-ionic dispersants can be: fatty alcohol polyglycol ethers, alkylphenol polyglycol ethers, fatty acid poly-glycol esters, fatty amide polyglycol ethers, fatty amine polyglycol ethers, alkoxylated triglycerides, (hydroxy)mixed ethers or mixed formals, alk(en)yl oligoglycosides, fatty acid N-alkylglucamides, protein hydrolysates, polyol fatty acid esters, sugar esters, sorbitan esters, polysorbates and amine oxides.

Particularly preferred non-ionic dispersants that may be mentioned are the alkyl and/or alkenyl oligoglycosides, which comply with the formula (I), R¹O-[G]p in which R¹ represents a linear or branched, saturated or unsaturated alkyl and/or alkenyl radical having 4 to 22 carbon atoms, G represents a sugar radical having 5 or 6 carbon atoms, preferably a glycoside radical and p represents numbers from 1 to 10 and preferably from 1 to 6.

These alkyl(ologo)glycosides can be obtained according to the appropriate processes of preparative organic chemistry. The alkyl and/or alkenyl oligoglycosides can be derived from aldoses or ketoses having 5 or 6 carbon atoms, preferably glucose. The preferred alkyl and/or alkenyl oligoglycosides are thus alkyl and/or alkenyl oligoglucosides. The index number p in the general formula (I) indicates the degree of oligomer-ization (DP), i.e. the dispersion of mono- and oligo-glycosides and represents a number between 1 and 10. Whereas p in a given compound must always be a whole number and here can especially assume the values p=1 to 6, the value p for a certain alkyl oligoglycoside is an analytically determined arithmetical variable, which is usually a fractional number. Preferably, alkyl and/or alkenyl oligoglycosides having an average degree of oligomerization p of 1.1 to 3.0 are employed. From the application technology point of view, those alkyl and/or alkenyl oligoglycosides are preferred whose degree of oligomerization is less than 1.7 and in particular lies between 1.2 and 1.4. The alkyl or alkenyl radical R can be derived from primary alcohols having 4 to 11, preferably 8 to 10, carbon atoms. Typical examples are butanol, caproyl, caprylyl, capryl and undecyl alcohol as well as their technical mixtures, as are obtained, for example, in the hydrogenation of technical fatty acid methyl esters or in the course of the hydrogenation of aldehydes from Roelen's oxo synthesis. Preferred alkyl oligo-glucosides are those of chain length C8-C10 (DP=1 to 3), which are obtained as a forerun in the distillative separation of technical C8-C18 coconut fatty alcohol and can be contaminated with a fraction of less than 6% by weight of C12-alcohol as well as alkyl oligo-glucosides based on technical C9/11 oxo alcohols (DP=1 to 3). The alkyl or alkenyl radical R can further also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol as well as their technical mixtures, which can be obtained as described above. Preferred alkyl oligoglucosides are those based on hardened C12/14-coconut alcohol having a DP of 1 to 3.

Further typical examples of suitable substances that alternatively form the component (b) are anionic surfactants that are selected from the group that is formed soaps, alkylbenzene sulfonates, alkane sulfonates, olefin sulfonates, alkyl ether sulfonates, glycerol ether sulfonates, [alpha]-methyl ester sulfonates, sulfo fatty acids, alkyl sulfates, alkyl ether sulfates, glycerol ether sulfates, hydroxy mixed ether sulfates, monoglyceride(ether)sulfates, fatty acid amide(ether)sulfates, mono- and dialkyl sulfosuccinates, mono- and dialkyl sulfo-succinamates, sulfotriglycerides, amide soaps, ether carboxylic acids and their salts, fatty acid isethionates, fatty acid sarcosinates, fatty acid taurides, N-acylamino acids, alkyl oligoglucoside sulfates, protein fatty acid condensates and alkyl(ether)phosphates.

Here, in particular alkyl ether sulfates have proven particularly advantageous that preferably comply with the formula (II): RO(CH₂CH₂O)ₙ—SO₃X (II), in which R represents a linear or branched, aliphatic alkyl and/or alkenyl radical having 6 to 22, preferably 12 to 18, carbon atoms, n represents numbers from 1 to 10, preferably 2 to 5 and X represents an alkali metal and/or alkaline earth metal, ammonium, alkylammonium, alkanolammonium or glucammonium. Typical examples of alkyl ether sulfates that can be used within the meaning of the invention are the sulfation products of addition products of an average 1 to 10 and in particular 2 to 5 mol of ethylene oxide to caproyl, caprylyl, capryl and 2-ethylhexyl alcohol, lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol and erucyl alcohol as well as their technical mixtures. The sulfation products can preferably be employed in the form of their alkali metal salts and in particular of their sodium salts.

In the case of the non-ionic dispersants, preferably sugar surfactants and particularly preferably surfactants from the class consisting of the alkyl polyglucosides can be used. The addition of these surfactants leads to a desired lowering of the viscosity of the dispersions and simultaneously improves the stability of the dispersions. In the case of the anionic surfactants, preferably surfactants from the class consisting of the fatty alcohol sulfates and particularly here particularly preferably long-chain fatty alcohol sulfates, such as, for example, sodium palmityl sulfate, sodium stearyl sulfate or mixtures of long-chain fatty alcohol sulfates, can be employed. Furthermore, for the adjustment of the viscosity small amounts of organic or inorganic salts, such as, for example, sodium sulfate, sodium sulfite, sodium cumenesulfonate or urea, can also be employed.

As waxes c), fundamentally all types of products solid at room temperature (21° C.) can be used, which were produced based on petrochemical or natural raw materials.

The waxes that are produced based on petrochemical raw materials include, for example, simple hydrocarbon compounds, such as paraffin waxes, or alternatively homopolymers produced by polymerization, such as polyethylenes, polyvinyl acetates, polyacrylates, oxidized homopolymers, such as, for example, oxidized polyethylenes, copolymers based on ethylene/acrylic acid, ethylene/propylene/maleic anhydride, ethylene/vinyl acetate or micronized polyethylene waxes. The melting point of these compounds is preferably between 40° C. and 160° C., particularly preferably between 60° C. and 140° C. Examples to be mentioned here would be some commercial products—again without a claim to completeness: AC330, AC 175, AC 5120, ACumist A12 from Honeywell, Licowax PE 130, Licowax PED 521, Licowax PED 192 from the company Clariant, Luwax OA3 wax from the company BASF. Furthermore, commercial products in which the polyethylene wax is already dispersed with anionic, non-ionic or cationic emulsifiers or dispersants in aqueous solutions, such as, for example, Polyquart® CCE from the company Cognis, can also be used.

However, as also described in the statement of the objective, waxes based on renewable raw materials, that is in particular waxes, which can be prepared, for example, by esterification, trans-esterification, etherification or amidation, are preferably used. Longer-chain fatty acids, fatty alcohols and fatty amines, preferably with C chains of C6-C22, for example, can be employed in these processes. Furthermore, however, it is also possible to combine long-chain fatty acids, fatty acid chlorides, fatty alcohols or fatty amines with short-chain, mono- or polybasic carboxylic acids, alcohols or amine compounds, amino-carboxylic acids, hydroxylamine compounds, that is, for example, ethanol, n-butanol, ethylene glycol, sorbitan, diethylene glycol, glycerol, triethanolamine, amino-ethylethanolamine. The melting point of these waxes is preferably between 20° C. and 120° C., particularly preferably between 30° C. and 80° C. To be mentioned as examples—without claim to completeness—would be: butyl stearate, cetyl palmitate, ethylene glycol distearate, glycerol monostearate, stearyl citrate, triethanolamine distearate, stearyl glutamate, di-n-cetyl ether and di-n-stearyl ether. Moreover, fatty acids, fatty alcohols or triglycerides, such as, for example, stearic acid, stearyl alcohol, hardened palm fat or naturally occurring hydrocarbon compounds, such as, for example, squalane can also be employed directly.

Typical examples of cationic dispersants are, in particular, tetraalkylammonium compounds, such as, for example, dimethyldistearylammonium chlorideldistearyl-ammonium chloride or hydroxyethyl/hydroxycetyl dimmonium chloride or “ester quats”.

In this case these are, for example, quaternized fatty acid triethanolamine ester salts or their alkoxylated derivatives. In addition to the quaternized fatty acid triethanolamine ester salts, possible ester quats are further also quaternized ester salts of fatty acids with diethanolalkylamines). Quaternized ester salts of fatty acids with 1,2-dihydroxypropyldialkylamines may be mentioned as a further group of suitable ester quats. Finally, suitable ester quats are additionally substances in which the ester bond is replaced by an amide bond. Such amide ester quats are obtainable on the market, for example, under the trademark Incroquat (Croda).

Examples of suitable amphoteric or zwitterionic surfactants are alkyl-betaines, alkylamidossines, aminopropionates, amino-glycinates, imidazolinium betaines and sulfossines. Examples of suitable alkylbetaines are the carboxy-alkylation products of secondary and in particular tertiary amines, such as, for example, the carboxy-methylation products of hexylmethylamine, hexyl-dimethylamine, octyldimethylamine, de-cyldimethyl-amine, dodecylmethylamine, dodecyldimethylamine, dodecylethylmethylamine, C12/14-coconut alkyldimethyl-amine, myristyldimethylamine, cetyldimethylamine, stearyldimethylamine, stearylethylmethylamine, oleyl-dimethylamine, C16/18-tallow alkyldimethylamine as well as their technical mixtures. Furthermore, carboxy-alkylation products of amidoamines are also suitable, thus, for example, reaction products of fatty acids with 6 to 22 carbon atoms, namely caproic acid, caprylic acid, capric acid, lauric acid, myristic acid, palmitic acid, palmoleic acid, stearic acid, isostearic acid, oleic acid, elaidic acid, petroselinic acid, linoleic acid, linolenic acid, elaostearic acid, arachic acid, gadoleic acid, behenic acid and erucic acid as well as their technical mixtures, with N,N-dimethylaminoethylamine, N,N-dimethylaminopropylamine, N,N-diethylaminoethylamine and N,N-diethylaminopropyl-amine, which are condensed with sodium chloroacetate. The use of a condensation product of C8/18-coconut fatty acid N,N-dimethylaminopropylamide with sodium chloro-acetate is preferred. Furthermore, imidazolinium betaines are also suitable. These substances are also known substances, which can be obtained, for example, by cyclizing condensation of 1 or 2 mol of fatty acid with polyacidic amines such as, for example, amino-ethylethanolamine (AEA) or diethylenetriamine. The corresponding carboxyalkylation products are mixtures of different open-chain betaines. Typical examples are condensation products of the abovementioned fatty acids with AEEA, preferably imidazolines based on lauric acid or in turn C12/14 coconut fatty acid, which are then betainized with sodium chloroacetate.

Fatty alcohols having at least 16 carbon atoms or polyol esters, however, are particularly preferred as dispersants. Preferred polyol esters contain as the polyol component sorbitans, or preferably glycerol, and as the acid fatty acids having 6 to 22 C atoms, where in particular unsaturated fatty acids can be preferred. Furthermore, natural or synthetic polymer dispersants can be used, e.g. sodium polyacrylates (Cosmedia® SP of the company Cognis) or products based on guar and xanthan gum. Furthermore, cationized starch or cellulose (e.g. Polyquaternium-10) are suitable dispersants (further commercial products are, for example, Polymer JR or the Dehyquart® guar of the company Cognis).

The textile treatment agents contain the hydroxylated lecithin preferably in amounts from 0.1 to 10% by weight, preferably from 1 to 8 and in particular from 1.5 to 5% by weight, based on the total weight of the composition. The dispersants are contained in amounts from 0.1 to 5% by weight, preferably from 0.2 to 2.5% by weight and in particular from 0.5 to 1% by weight, based on the total weight of the composition, and waxes in amounts from 1 to 15% by weight, preferably from 2 to 10% by weight and in particular from 2.5 to 5% by weight, based on the total weight of the composition.

The compositions of the present invention are always aqueous (preferably completely deionized water is selected) and preferably contain the water in amounts from 65 to 98% by weight, preferably from 80 to 95% by weight and in particular from 85 to 95% by weight, based on the total weight of the composition.

The textile treatment agents can furthermore additionally contain additives e), selected from the group consisting of organic and/or inorganic salts (for example sodium chloride, sodium sulfate, sodium cumenesulfonate or urea), emulsifiers, preservatives (e.g. benzoic acid), colorants, fragrances, polymers, non-aqueous solvents and acids or bases. The latter are used to adjust the aqueous compositions to a desired pH, which preferably between 2.0 and 8.0 and in particular from 3.0 to 7.0, where a value between 3.0 and 4.0 can preferably be selected.

The additives e) are then preferably contained in amounts from 0.01 to 10.0% by weight, preferably from 0.1 to 5.0% by weight and in particular from 1.0 to 5.0% by weight, based on the total weight of the composition.

The textile treatment compositions are preferably free of cationic textile softeners, starch and starch derivatives, liposomes and/or non-hydroxylated lecithin. Preferably, the compositions can be free of cationic textile softeners, which in particular are selected from the group consisting of quaternized fatty acid triethanolamine ester salts, such as, for example, are actually described in EP 848 103 A2, see here in particular the structures according to the general formula (I), (II) and (III) in the claims of the specification and according to the description in on page 4, line 11 to page 5, line 32.

In a further embodiment, the compositions consist only of the ingredients a)-d) according to the above description.

The production of the compositions takes place in a manner known per se by mixing the components and then heating with stirring. The components are preferably heated to a temperature above the melting point of the wax component, in particular the temperature ranges from 60 to 80° C. is thus to be selected. After about 20 to 40 min, a stable dispersion is then formed, which is cooled and optionally additionally adjusted to a desired pH with inorganic or organic acids or bases. However, it is also possible to employ waxes pre-dispersed in water, e.g. an aqueous polyethylene dispersion or waxes based on carboxylic acid esters (e.g. Polyquart® CCE or Plantatex® HCC, both from

Cognis), which can then directly be stirred together with the other components.

The compositions or the hydroxylated lecithins are preferably used for imparting soft handle in textile fibers and fabrics produced therefrom, in particular those of wool or cotton, but also for keratinic fibers (e.g. hair). The use, however, also includes textiles or fibers that contain synthetic fibers (“mixed fabric”) or consist of synthetic fibers. Thus hydroxylated lecithin is preferably suitable for the production of detergents, washing aftertreatment agents, but also for the production of cosmetic agents, in particular for shampoos.

The compositions according to the present description can be used, for example, as textile treatment compositions in a washing process for textiles, the compositions being brought into contact with the textiles, preferably in a domestic washing machine. Here, the hydroxylated lecithin, preferably in the form of a wax-containing composition as described above, can be added before, during or after the actual washing operation. It may be advantageous here to increase the temperature of the water during the bringing into contact, e.g. to values between 30 and 60° C.

### EXAMPLES

As an example, 4 different formulations were prepared. For the preparation of the lecithin-containing dispersions, all components were introduced into a beaker and heated to 75° C. with stirring. The mixture was subsequently stirred at this temperature for 30 min and then cooled to room temperature again with stirring. The viscosity was then determined using a Brookfield viscometer (20° C., spindle 3, 20 rpm).

The dispersions thus prepared had the following composition (data always in % by weight, remainder to 100% by weight: water):

| | Formu- | Formu- | Formu- | |
|---|---|---|---|---|
| | lation 1 | lation 2 | lation 3 | Formulation 4 |
| Lanette ® O | 4 | | | |
| Dehymuls ® SMS | | 4 | | |
| Cutina ® GMS-V | | | 4 | 4 |
| Lanette ® E | 1 | 1 | 1 | 1 |
| Hydroxylated | 2 | 2 | 2 | 2 |
| soya lecithin | | | | |
| Glucopon ® 425 | | | 0.5 | |
| N/NH | | | | |
| Glucopon ® 600 CS | 0.5 | 0.5 | | 0.5 |
| UP | | | | |
| Urea | | | 0.1 | 0.1 |
| Benzoic acid | 0.5 | 0.5 | 0.5 | 0.5 |
| pH | 3-4 | 3-4 | 3-4 | 3-4 |
| Viscosity [mPaS] | 50 | 25 | 100 | 50 |

Lanette® O is a mixture of palmityl and stearyl alcohol of Cognis, Dehymuls® SMS is the sorbitan monostearate of Cognis, Cutina® GMS-V is a glycerol monostearate of Cognis, Lanette E is a sodium palmityl sulfate of Cognis, the hydroxylated soya lecithin was a product of the Solae company with the name Solec® A, Glucopon® 425 N/NH and Glucopon® 600 CS UP are alkyl polyglycosides of Cognis. Commercially available benzoic acid (Sigma/Aldrich) was used by way of example as a preservative, as well as urea (Sigma/Aldrich) for the adjustment of the viscosity.

For the comparison of the softening power to be achieved therewith, a dispersion containing 15% by weight of a commercially available cationic softener (Dehyquart® AU 46 of Cognis) was prepared. Using these products, washing tests were carried out and the washed textiles were compared to each other using an objective method for the assessment of the softness/smoothness.

1) Washing Experiments with Various Concentrations of Ester Quat.

2000 g each of ballast washing of cotton per machine were prewashed three times at 95° C. using 75 g each of ECE-2 (supplier: wfk/Krefeld) for the removal of the dressing and additionally rinsed three times at 95° C. without detergent. These laundry items were washed at 40° C. with ECE-2 and in each case different amounts of Dehyquart® AU 46 were added in the rinsing operation. The washing was then dried and small rectangular strips were punched out from a small section. These strips were air-conditioned and subsequently drawn through a special arrangement of metallic bars by means of with a pinch machine for recording of force/path graphs.

The lower the force for pulling through the strips, the smoother/softer and more flexible the textile fabric. This method is comparable with known methods for the measurement of the wet and dry combability of hair. Experiments for method validation carried out beforehand showed a good agreement with sensory data, which were determined by human subjects on a monadic scale for the assessment of smoothness, softness and suppleness. The following values were determined here:

| | Machine 1 | Machine 2 | Machine 3 | Machine 4 | Machine 5 |
|---|---|---|---|---|---|
| Amount of | — | 2.4 g | 12 g | 24 g | 36 g |
| 15% by | (only | | | (standard | |
| weight | rinsing | | | amount) | |
| dispersion | with | | | | |
| of | water) | | | | |
| Dehyquart ® | | | | | |
| AU 46 | | | | | |
| Force [N] | 1.66 +/− 0.13 | 1.48 +/− 0.14 | 1.22 +/− 0.06 | 1.00 +/− 0.08 | 0.83 +/− 0.16 |

It is seen that with an increasing amount of cationic softener the force for pulling through the strips is lowered, that is the textiles become—as was to be expected—smoother/softer and more supple.

### 2) Washing Experiments Using the Lecithin-Containing Wax Dispersions

Analogously to the washing experiments described under 1), the lecithin-containing wax dispersions described above were added in the last rinsing operation of the machine. For comparison, rinsing was carried out only with water and the standard amount of cationic softener. After analogous treatment, the following data were determined:

| | Machine 1 | Machine 2 | Machine 3 | Machine 4 | Machine 5 | Machine 6 |
|---|---|---|---|---|---|---|
| Amount of | — | 24 g of ester | 48 g of | 48 g of | 48 g of | 48 g of |
| softener | (only rinsing | quat, 15% strength | formulation 1 | formulation 2 | formulation 3 | formulation 4 |
| | with water) | (standard amount) | 7% strength | 7% strength | 7% strength | 7% strength |
| Force [N] | 1.94 +/− 0.29 | 1.08 +/− 0.06 | 1.09 +/− 0.06 | 1.13 +/− 0.04 | 0.96 +/− 0.05 | 0.96 +/− 0.01 |

It is seen that all lecithin-containing wax dispersions achieve similar low values for the force as the ester quat-containing dispersion at equal concentration. The lecithin-containing wax dispersions according to the invention thus achieve similar, in some cases even better, powers with respect to smoothness, softness and suppleness of textile fabrics.

1. A softening treatment composition, comprising:
a) hydroxylated lecithin;
b) at least one dispersant;
c) at least one wax; and
d) water;
wherein the composition is effective to provide sensory properties to textiles or keratin-containing fibers.

2. The softening treatment composition as claimed in claim 1, wherein the at least one dispersant comprises a compound of the formula (I) R¹—O-[G]ₚ are selected, where R¹ represents a linear or branched, saturated or unsaturated alkenyl or alkyl radical having 4 to 22° C. atoms, G denotes a glycoside radical and p can assume values from 1 to 6.

3. The softening treatment composition as claimed in claim 1, wherein the at least one wax comprises a renewable raw materials.

4. The softening treatment composition as claimed in claim 1, wherein the at least one wax comprises a fatty alcohol having at least 16 carbon atoms or polyol esters.

5. The softening treatment composition as claimed in claim 1, wherein the hydroxylated lecithin comprises soya, rapeseed oil, or egg lecithin comprises soya, rapeseed oil, or egg lecithin.

6. The softening treatment composition as claimed in claim 1, wherein the composition comprises the hydroxylated lecithin in an amount from 0.1 to 10% by weight, based on the total weight of the composition.

7. The softening treatment composition as claimed in claim 1, wherein the composition comprises the one or more dispersants in an amount from 0.1 to 5% by weight.

8. The softening treatment composition as claimed in claim 1, wherein the composition comprises the at least one wax in an amount from 1 to 15% by weight.

9. The softening treatment composition as claimed in claim 1, wherein the composition comprises water in an amount from 65 to 98% by weight.

10. The softening treatment composition as claimed in claim 1, wherein the composition has a kinematic viscosity (measured at 20° C. using a Brookfield viscometer, spindle 3, 20 rpm) of between 15 and 250 mPa s.

11. The softening treatment composition as claimed in claim 1, wherein the composition further comprises one or more additives e), selected from the group consisting of organic and/or inorganic salts, emulsifiers, preservatives, colorants, fragrances, polymers, non-aqueous solvents and acids or bases.

12. The softening treatment composition as claimed in claim 11, wherein the additives e) are in an amount from 0.01 to 10.0% by weight.

13. The softening treatment composition as claimed in claim 1, wherein the composition is free of cationic textile softeners, liposomes and/or non-hydroxylated lecithin.

14. A softening treatment composition, consisting of:
a) hydroxylated lecithin;
b) at least one dispersant;
c) at least one wax; and
d) water;
wherein the composition is effective to provide sensory properties to textiles or keratin-containing fibers.

15. A method for softening of textiles or keratin-containing fibers, the method comprising obtaining the softening treatment composition of claim 1, and applying the softening treatment composition to the textiles or the keratin-containing fibers.

16. The method of claim 15, wherein the keratin-containing fibers comprise hair and the softening treatment composition is delivered by detergents, washing aftertreatment agents, or cosmetic preparations.

17. The method of claim 15, wherein the softening treatment is applied to the textiles in a washing process, with the aid of a domestic washing machine.

18. A method for softening of textiles or keratinic-containing fibers, the method comprising obtaining the softening treatment composition of claim 14, and applying the softening treatment composition to the textiles or the keratin-containing fibers.
